(19) United States
(12) Patent Application Publication
(10) Pub. No.: US 2008/0234569 A1
(43) Pub. Date: Sep. 25, 2008

(54) Mri Probe for Prostate Imaging

(75) Inventors: Gil Tidhar, Modiin (IL); Aharon Blank, Kfar-Vradim (IL)

Correspondence Address: MARTIN D. MOYNIHAN d/b/a PRTSI, INC., P.O. BOX 16446, ARLINGTON, VA, 22215, US

(73) Assignee: Topspin Medical (Israel) Ltd., Lod (IL)

(21) Appl. No.: 10/597,325

(22) PCT Filed: Jan. 20, 2005
(86) PCT No.: PCT/IL2005/000074

§ 371 Date: May 19, 2008

Related U.S. Application Data
(60) Provisional application No. 60/537,030, filed on Jan. 20, 2004.

(51) Int. Cl. A61B 5/055; A61B 8/00

(52) U.S. Cl. 600/423; 600/459

(57) ABSTRACT

A rectal probe adapted for ultrasound and magnetic resonance imaging of the prostate, comprising: a) an ultrasound imaging probe; b) an MRI probe comprising a first magnetic field source for creating a static magnetic field in an MRI imaging region outside the rectal probe, a second magnetic field source for creating a time-varying magnetic field which excites nuclei in the MRI imaging region, and a receiver for receiving NMR signals from the excited nuclei and generating MRI imaging data indicative thereof; and c) a link joining the ultrasound probe and the MRI probe.

### RELATED APPLICATIONS

The application claims the benefit under 119(e) of U.S. patent application No. 60/537,030 entitled “MRI Probe for Prostate Imaging”, filed on Jan. 20, 2004, the disclosure of which is incorporated herein by reference.

### FIELD OF THE INVENTION

The field of the invention is medical imaging, especially magnetic resonance imaging of the prostate.

### BACKGROUND OF THE INVENTION

Early detection of prostate cancer is important for successful treatment. The most common methods of screening for prostate cancer, manual examination and blood tests for PSA, fail to detect some malignant tumors, and sometimes give false positives. Biopsy is a definitive way of detecting a tumor and evaluating how dangerous it is. It usually employs a small point sampling by sharp long syringe. Because blood tests give no indication of where in the prostate a tumor is located, and manual exams give only a rough idea, biopsies often miss a tumor. To avoid this, multiple biopsies may be made in different parts of the prostate, but this can cause greater patient discomfort, and may miss a small or diffusive tumor anyway.

Various medical imaging technologies have been used or suggested for detecting and precisely locating prostate cancer, as well as for guiding biopsies, and for treatment of prostate cancer, for example by radiation, including implantation of radioactive sources in the prostate, and by thermal ablation using radio waves, microwaves, or ultrasound. See, for example, U.S. Pat. Nos. 6,371,903; 5,404,881 to Cathaud et al; U.S. Pat. No. 6,425,867 to Vaezy et al; U.S. Pat. Nos. 6,432,067; 6,402,742 to Blewett et al; U.S. Pat. Nos. 6,311,084; and 6,129,670, the disclosures of which are incorporated herein by reference. Ultrasound imaging is inexpensive, and can be done with a transrectal ultrasound (TRUS) probe, which is brought close to the prostate. When the ultrasound imaging is used to guide ultrasound or RF ablation of prostate tissue, the rectal probe can also contain temperature sensors, or RF field sensors, to provide feedback for the ablation, as described by Cathaud et al. But ultrasound imaging has limited ability to distinguish normal from malignant tissue.

Conventional MRI, in which the patient is placed within the bore of a large magnet, is better at distinguishing between different types of soft tissue, including normal and malignant prostate tissue, but is expensive. This is also true if the MRI receiver is located in a rectal probe close to the prostate, in order to improve the signal to noise ratio (SNR), as described, for example, in U.S. Pat. Nos. 5,170,789; 6,549,800 to Atalar et al; U.S. Pat. No. 6,470,204 to Uzgiris et al; and U.S. Pat. No. 5,451,232, the disclosures of which are incorporated herein by reference. U.S. Pat. No. 5,810,007, to Holupka, the disclosure of which is incorporated herein by reference, describes software for fusing an ultrasound image of the prostate, obtained with a rectal probe, and a conventional MRI image of the prostate. The fused image incorporates information from both images, and is particularly useful for monitoring treatment of the prostate.

U.S. Pat. No. 5,572,132, to Pulyer, describes a self-contained MRI probe, including a permanent magnet and an RF coil used for transmitting MRI pulses as well as for receiving MRI signals. Such a probe can be used in the rectum for prostate imaging, as well as in other body cavities, and would be much less expensive than conventional MR. However, the requirement that this kind of probe have a relatively homogeneous static magnetic field in the imaging region, limits the magnetic field strength that can be obtained in the imaging region, and hence limits the SNR or the resolution. Blank et al, in U.S. Pat. No. 6,704,594, describes a self-contained MRI probe. This probe uses pulse sequences that do not require such high magnetic field homogeneity in the imaging region, and hence may be capable of better spatial resolution for a given SNR and image acquisition time. The disclosures of Pulyer and of Blank et al are incorporated herein by reference.

### SUMMARY OF THE INVENTION

An aspect of an embodiment of the invention concerns a rectal probe for prostate imaging which incorporates both a transrectal ultrasound (TRUS) probe, and a self-contained MRI probe. Optionally, the MRI probe is a self-contained MRI probe of the type which does not require a very homogeneous magnetic field in the imaging region. In one example, the MRI probe is a Topspin MRI (TMRI) probe. The TMRI probe is similar to a scaled-up version of the self-contained intravascular MRI probes designed by Topspin Medical Israel, Ltd., and described, for example, in U.S. Pat. No. 6,704,594, but with differences, for example z-gradient coils, which make it more suitable for prostate imaging. Some embodiments of the present invention are described herein using the TMRI probe for convenience. However, the use of this probe is meant to be non-limiting, and other designs can be used. Optionally, the TRUS and TMRI probes are arranged longitudinally, with a flexible link between them, allowing the two probes to conform to the natural curvature of the rectum. The ultrasound image, which is acquired in a relatively short time, typically no more than a few seconds, and accurately shows the boundaries of the prostate, is optionally used to aim the TMRI probe in a direction so that the field of view covers the prostate but is not wider than necessary. Since the MRI data typically takes several minutes to acquire, it is useful to be able to aim the TMRI probe correctly the first time, and not to waste time acquiring MRI data outside the prostate. It is primarily the MRI data that distinguishes normal from malignant tissue, and different stages of malignant tissue. Optionally, the degree and direction of bending of the flexible link between the TMRI probe and the TRUS probe are controllable, so that the TMRI probe may be aimed relative to the TRUS probe. Additionally or alternatively, the field of view of the TMRI probe is adjustable by software, for example by adjusting the relative phases of two or more RF antennas. Optionally, such adjustment is applied in real-time, for example to correct for motions, which motions are optionally detected on using the ultrasound probe.

Optionally, the ultrasound image acquired by the TRUS probe, which may show the boundary of the prostate at higher resolution than the MRI image, is combined with the MRI image, which distinguishes malignant and normal tissue better than the ultrasound image. The combined image may be more useful than either image by itself, for example for purposes of advance planning of where to direct biopsies or therapy (including surgery), and/or for example, for purposes of guiding biopsies or therapy in real time. Accurate three-dimensional knowledge of the location and boundaries of tumors optionally leads to a better rate of success in treating the cancer and/or optionally leads to lower rates of complications from surgery or other therapy, since healthy tissue will be disturbed as little as possible.

An aspect of an embodiment of the invention concerns a TMRI probe, used in the rectum for prostate imaging. TMRI probes, because they have a highly inhomogeneous magnetic field, are more sensitive to diffusion of protons (essentially diffusion of water molecules) than conventional MRI, or than a Pulyer-type MRI probe which requires a relatively homogeneous magnetic field. The increased sensitivity to diffusion optionally allows the probe to more accurately distinguish normal and malignant prostate tissue, and/or distinguish different stages of malignancy.

In general, MRI is more sensitive to diffusion in a highly inhomogeneous magnetic field, because the excited nuclei diffuse out of resonance more quickly. The effect of diffusion on the MRI signal in an inhomogeneous field is to reduce the MRI echo signal. This is similar to the effect of the transverse spin relaxation time, T₂, which is due to diffusion in slight field inhomogeneities on a molecular scale. In conventional MRI, or even in MRI with a Pulyer-type probe, the magnetic field is so homogeneous that the reduction in echo signal due to diffusion during a T₂ time scale, is small. Hence differences in diffusion coefficient of different types of tissue within the examined sample are difficult to measure, especially when these different tissues also have different T₂ values. With a highly inhomogeneous magnetic field, such as that produced by a TMRI probe, the reduction in echo signal will, in some implementations, be dominated by diffusion effects, and not T₂. This optionally enables more precise measurement of the diffusion coefficient of the various tissues, for some embodiments, at least partially independent of T₂.

Potential advantages of using a TMRI probe are one or more of a) the increased ability to distinguish different types of tissue may make the probe useful in guiding biopsies or therapy; b) a TMRI probe may be used in a urologist's clinic; and c) using a TMRI probe is likely to be less expensive than using conventional MRI. Optionally, the TMRI probe is used in conjunction with a TRUS probe, which produces ultrasound images in real time, in which the boundary of the prostate is clearly visible, but in which normal and malignant tissue within the prostate are not well distinguished. By combining the MRI and ultrasound images, one can obtain an image of the prostate in real time in which the malignant regions are clearly visible. This may be useful for accurately guiding a biopsy or a therapeutic procedure, during which the prostate may move.

The combination of MRI and ultrasound images can be more useful for determining the stage of development of prostate cancer than either MRI or ultrasound images alone. For example, the capsule surrounding the prostate may be seen clearly in the ultrasound image. If the MRI image reveals a growing tumor pressing against the capsule, then the ultrasound image may reveal whether the capsule is still intact (T2 stage cancer), or has been penetrated by the tumor (T3 stage cancer), and appropriate treatment may be chosen.

An aspect of an embodiment of the invention concerns a self-contained MRI probe, such as a TMRI probe, used in the rectum for prostate imaging, in which an inflatable balloon attached to the probe presses the probe against the anterior wall of the rectum, so that it will be as close to the prostate as possible.

An aspect of an embodiment of the invention concerns methods of using a self-contained MRI probe, such as a TMRI probe, in the rectum for prostate imaging, in which a number of images, each with relatively low SNR, are obtained sequentially, and are combined to make an image with higher SNR. Because the prostate may move in an unpredictable way, by as much as 15 mm, relative to the MRI probe, during the time that the different images are acquired, software is optionally used to align the different images before combining them, compensating for the motion.

Optionally, there is also a TRUS probe adjacent to the MRI probe, which produces relatively high SNR images of the prostate in a short enough time so that the prostate does not move very much (but without much ability to distinguish malignant and normal tissue), while the MRI probe is acquiring MRI imaging data. The ultrasound images are optionally used to align the different MRI images, and the aligned MRI images are combined to produce the high SNR MRI image, which can distinguish malignant and normal tissue. Optionally, instead of or in addition to using the software to align the MRI images, the alignment is done by changing the field of view of the MRI probe in real time, while the MRI probe is acquiring the imaging data, in response to information in the ultrasound images. Alternatively or additionally, the TRUS probe is used to detect periodic movements which are corrected for when processing the MRI data.

Optionally, even without an ultrasound probe, the different MRI images are aligned, by the software, by finding a displacement for each MRI image which maximizes the sharpness of features in the combined image, or some other characteristic of the combined image. Optionally, the software not only corrects for the relative displacement of the prostate between two images acquired at different times, but also calculates the velocity of the prostate during MRI image acquisition, and corrects for motion artifacts in the MRI image. The velocity may be found, for example, by comparing the displacement in different images, or directly by Doppler measurements during the ultrasound imaging.

There is thus provided, in accordance with an embodiment of the invention, a rectal probe adapted for ultrasound and magnetic resonance imaging of the prostate, comprising:
a) an ultrasound imaging probe;
b) an MRI probe comprising a first magnetic field source for creating a static magnetic field in an MRI imaging region outside the rectal probe, a second magnetic field source for creating a time-varying magnetic field which excites nuclei in the MRI imaging region, and a receiver for receiving NMR signals from the excited nuclei and generating MRI imaging data indicative thereof; and
c) a link joining the ultrasound probe and the MRI probe.

Optionally, the link is flexible, thereby allowing a direction of orientation of the MRI probe to vary relative to a direction of orientation of the ultrasound probe.

Optionally, the MRI probe has a longitudinal axis, and the static magnetic field is substantially inhomogeneous, defined as monotically decreasing with increasing distance from the longitudinal axis, outside the MRI probe, in all directions.

Optionally, the first magnetic field source is a permanent magnet, and the magnetic field is at least 0.35 tesla at at least one point on the surface of the MRI probe.

Optionally, the first magnetic field source is a permanent magnet, and the magnetic field is at least 0.01 tesla at at least one point at a distance 50 mm from the surface of the MRI probe.

Optionally, the first magnetic field source is a permanent magnet, and the magnetic field has a gradient of at least about one tesla per meter, at at least one point 50 mm from the surface of the MRI probe.

Optionally, the second magnetic field source is a coil which produces a magnetic field of at least 4 micro-tesla per ampere of current, at at least one point at a distance 50 mm from the surface of the probe.

There is further provided, according to an embodiment of the invention, an imaging system comprising:
a) a rectal probe according to an embodiment of the invention; and
b) an RF power supply which supplies power to the second magnetic field source;
   wherein the RF power supply is capable of supplying the second magnetic field source with sufficient power at a great enough frequency range to simultaneously excite nuclei in the imaging region to generate NMR signals having a frequency bandwidth of more than 5% of their mean frequency, and wherein the static magnetic field monotonically decreases with increasing radial distance from the outer surface of the MRI probe, everywhere outside the outer surface of the MRI probe.

Optionally, the NMR signals have a frequency bandwidth of more than 10% of their mean frequency.

Optionally, the NMR signals have a frequency bandwidth of more than 20% of their mean frequency.

There is further provided, according to an embodiment of the invention, an imaging system comprising:
a) a rectal probe according to and embodiment of the invention; and
b) an RF power supply which supplies a given power to the second magnetic field source;
   wherein the MRI probe, when supplied with said given power by the RF power supply, is capable of producing MRI imaging data with sufficient spatial resolution and signal to noise ratio to be capable of revealing a tumor 5 mm in diameter located anywhere in the prostate.

Additionally or alternatively, the second magnetic field source, when supplied with said given power by the RF power supply, is capable of creating a time varying magnetic field of at least 0.0025 tesla at at least one point at a distance of 50 mm from the surface of the MRI probe.

Optionally, the imaging system also comprises a controller which controls the RF power supply to produce a timed sequence of RF pulses, wherein the controller is adapted to control the RF power supply to produce the sequence of RF pulses with a repetition rate greater than one pulse every 0.5 milliseconds and less than one pulse every 0.25 milliseconds, and the RF power supply is capable of producing the sequence of RF pulses at said repetition rate.

There is further provided, according to an embodiment of the invention, an imaging system comprising:
a) a rectal probe according to an embodiment of the invention, with a longitudinal axis, also including at least one gradient coil which produces a magnetic field gradient in the direction of the longitudinal axis, or in an azimuthal direction around the longitudinal axis;
b) a gradient coil power supply which supplies current to the gradient coil; and
c) a controller which controls the gradient coil power supply to produce a timed sequence of gradient pulses;
   wherein the controller is adapted to make the gradient pulses at least about 0.2 milliseconds long.

There is further provided, according to an embodiment of the invention, a method of imaging the prostate, the method comprising:
a) inserting a rectal probe according to claim 1 into a rectum;
b) acquiring at least one ultrasound image of the prostate, using the ultrasound probe;
c) acquiring MRI imaging data of the prostate using the MRI probe; and
d) reconstructing an image of the prostate from the MRI imaging data, using an image reconstruction procedure;

wherein an adjustment is made to one or both of the MRI probe and the image reconstruction procedure using information from the ultrasound image.

Optionally, the adjustment causes the MRI imaging region to correspond more closely to the prostate or to a desired portion of the prostate.

Optionally, the at least one ultrasound image comprises a plurality of ultrasound images acquired at different times between the beginning and end of acquisition of the MRI image, and the adjustment corrects for motion of the prostate occurring between the beginning and end of acquisition of the MRI image.

Optionally, the information from the ultrasound image comprises a Doppler shift, and the adjustment corrects for motion of the prostate occurring during acquisition of the MRI image.

There is further provided, according to an embodiment of the invention, a method of diagnosing the stage of prostate cancer, comprising:
a) inserting a rectal probe according to claim 1 into a rectum;
b) acquiring an ultrasound image of the prostate using the ultrasound probe;
c) acquiring an MRI image of the prostate using the MRI probe;
d) finding prostate cancer on the MRI image; and
e) examining the ultrasound image to determine whether or not the prostatic capsule is intact in the vicinity of the prostate cancer found on the MRI image.

Optionally, acquiring an MRI image comprises acquiring a diffusion-weighted MRI image.

There is further provided, according to an embodiment of the invention, a method of performing one or both of a biopsy and therapy on the prostate, comprising:
a) inserting a rectal probe according to claim 1 into a rectum;
b) acquiring at least one ultrasound image of the prostate, using the ultrasound probe;
c) acquiring MRI imaging data of the prostate using the MRI probe;
d) reconstructing an MRI image of the prostate from the MRI imaging data, using an image reconstruction procedure; and
e) performing one or both of a biopsy and therapy on the prostate, guided in real time by the ultrasound image and the MRI image.

There is further provided, according to an embodiment of the invention, a method of diagnosing prostate cancer, comprising:
a) inserting into the rectum an MRI probe with a longitudinal axis, the MRI probe comprising a first magnetic field source for creating a substantially inhomogeneous static magnetic field in an MRI imaging region outside the probe, a second magnetic field source for creating a time-varying magnetic field which excites nuclei in an extended sub-region of the MRI imaging region, and a receiver for receiving the NMR signals from the excited nuclei and generating MRI imaging data indicative thereof;
b) acquiring a diffusion-weighted MRI image of the prostate using the MRI probe; and
c) finding prostate cancer on the MRI image by using the difference in diffusion rate between cancerous and healthy tissue.

There if further provided, according to an embodiment of the invention, an imaging system comprising:
a) an MRI rectal probe adapted for imaging of the prostate, the probe having a longitudinal axis and comprising a first magnetic field source for creating a static magnetic field in an MRI imaging region outside the probe, a second magnetic field source for creating a time-varying magnetic field which excites nuclei in the MRI imaging region, and a receiver for receiving NMR signals from the excited nuclei and generating MRI imaging data indicative thereof; and
b) an RF power supply which supplies power to the second magnetic field source;
   wherein the RF power supply is capable of supplying the second magnetic field source with sufficient power at a great enough frequency range to simultaneously excite nuclei in the imaging region to generate NMR signals having a frequency bandwidth of more than 5% of their mean frequency, and wherein the static magnetic field monotonically decreases with increasing radial distance from the longitudinal axis, everywhere outside the probe.

There is further provided, according to an embodiment of the invention, an MRI probe having a longitudinal axis, the probe comprising:
a) a first magnetic field source for creating a static magnetic field in an MRI imaging region outside the probe, which static magnetic field monotonically decreases with increasing distance from the longitudinal axis to the probe, everywhere outside the probe;
b) a second magnetic field source for creating a time-varying magnetic field which excites nuclei in the MRI imaging region;
c) a receiver for receiving the NMR signals from the excited nuclei and generating MRI imaging data thereof; and
d) at least one z gradient coil which produces a magnetic field gradient in the direction of the longitudinal axis.

Optionally, the probe is of a size suitable for use as a rectal probe for imaging the prostate.

### BRIEF DESCRIPTION OF THE DRAWINGS

Exemplary, non-limiting, embodiments of the invention are described in the following sections with reference to the drawings. The drawings are generally not to scale and the same or similar reference numbers are used for the same or related features on different drawings.

FIG. 1 is a cut-away view of a combined TMRI and TRUS probe, in place inside the body, as well as a schematic view of a controller and power supplies, according to an exemplary embodiment of the invention;

FIG. 2A is a cut-away view of a TMRI probe in place inside the body, according to another exemplary embodiment of the invention;

FIG. 2B is a cut-away view of a TMRI probe in place inside the body, according to another exemplary embodiment of the invention;

FIGS. 3A-3D and 4A-4C show magnet and coil configurations in accordance with exemplary embodiments of the invention;

FIG. 5A is perspective side view of a TMRI probe in accordance with an alternative embodiment of the invention;

FIG. 5B is a top cut-away view of the TMRI probe shown in FIG. 5A, with the top magnet removed;

FIG. 5C is a perspective side view of the TMRI probe shown in FIG. 5A, showing the field of view;

FIG. 6 is a flowchart showing a method of using combined ultrasound and MRI imaging data to reconstruct images of the prostate, according to an exemplary embodiment of the invention;

FIG. 7 schematically shows a set of images reconstructed at different stages, according to the method shown in FIG. 6; and

FIG. 8 schematically shows two images of the prostate, reconstructed according to the method of FIG. 6, with T2 and T3 stage tumors.

### DETAILED DESCRIPTION OF EXEMPLARY EMBODIMENTS

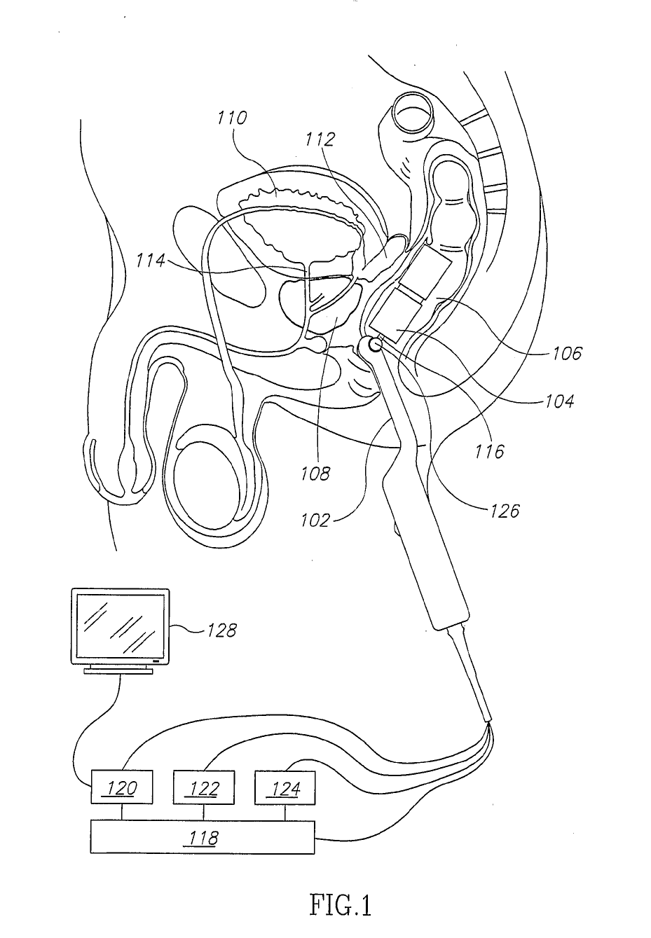

FIG. 1 shows a transrectal ultrasound (TRUS) probe 102, optionally coupled to a Topspin magnetic resonance imaging (TMRI) probe 104, inserted into the rectum 106, for imaging the prostate 108, in accordance with an exemplary embodiment of the invention. The bladder 110, seminal vesicles 112, and urethra 114 are also shown. TMRI probe 104 is a self-contained MRI probe, with its own permanent magnets, gradient coils, and RF antenna for transmitting NMR pulse sequences and receiving NMR data signals. Unlike some other self-contained MRI probes, such as the probe described by Pulyer, the TMRI probe is designed to produce a substantially inhomogeneous static magnetic field in the imaging region, and simultaneously excites nuclei over a substantial range of magnetic field strengths, to produce NMR signals with a substantial bandwidth. For example, the bandwidth is more than 5% of the mean frequency of the signals, or more than 10%, or even more than 20%, at least for part of the imaging region. One or more cables, not shown in the drawing, which carry RF power to the TMRI probe, and carry NMR data from the TMRI probe, are optionally routed along or through the TRUS probe. Optionally, the one or more cables also carry control signals to the TMRI probe, for example to tune the RF antenna or to adjust its direction of sensitivity.

Optionally, the TRUS probe is a standard off-the-shelf TRUS probe, and the TMRI probe is adapted to be joined to it. Alternatively, the TRUS probe is specially designed to be used with the TMRI probe. Optionally there is a flexible link 116 between the TRUS probe and the TMRI probe, which allows the relative orientation of the two probes to conform to the curvature of the rectum, and also allows the direction of orientation of the TMRI probe to be adjusted to optimize the field of view for imaging the prostate. Optionally, this adjustment is made in response to imaging data from the TRUS probe. It may be advantageous to give the flexible link two degrees of freedom, like a ball and socket joint, particularly if the flexible link is used to adjust the orientation of the TMRI probe. If the flexible link is only used to allow the two probes to conform to the curvature of the rectum, then a hinge-like joint with only one degree of freedom may be sufficient. Alternatively, link 116 is not flexible at all, but is at a fixed angle which conforms to the curvature of the rectum for example. However, making link 116 flexible may make it easier to insert the probe in the rectum.

Optionally, there is an element 126 which controls the angle of link 116, or both angles if it has two degrees of freedom, using a motor, for example. Optionally, element 126 also senses the angle (or angles) of link 116, providing feedback for controlling the angle. Alternatively, element 126 only senses the angle (or angles) of link 116, and does not control the angle. The angle of link 116 might still be controlled passively by adjusting the position of the probe in the rectum, for example. Even if the angle of link 116 is not controlled at all, sensing the angle is still useful, for example, if the ultrasound images are being used to compensate for motion artifacts in the MRI image.

Optionally, the positions of the TRUS and TMRI probes are reversed from that shown in FIG. 1. However, having the TRUS probe in the more proximal position, and the TMRI probe in the more distal position, as shown in FIG. 1, has the potential advantage that the TRUS probe can be used in the usual position for a TRUS probe by itself, and hence a standard TRUS probe, or a TRUS probe with only minor changes in design, can be used.

The TMRI probe is significantly closer to the peripheral zone of the prostate than to the transient and central zones. Because the static and RF magnetic fields are greater near the probe, and the antenna is more sensitive to signals emitted near the probe, the image typically has higher resolution and/or higher SNR in the peripheral zone of the prostate. For purposes of detecting cancer, this is suitable, since 75% of prostate cancers originate in the peripheral zone, and the vast majority of capsular invasion takes place in the direction of the peripheral zone, for example into the seminal vesicles. However, in an exemplary embodiment of the invention, the probe is designed to have adequate resolution and SNR even in the part of the prostate furthest from the probe, so that, for example, tumors at least 5 mm in diameter can be detected anywhere in the prostate.

A controller 118 controls the pulse sequences for the TMRI probe, by controlling an RF power supply 120 and a gradient coil power supply 122. Controller 118 also controls the output of the TRUS probe, by controlling TRUS power supply 124. Controller 118 also receives and analyzes imaging data from the TMRI and TRUS probes. Optionally, images reconstructed by controller 118 from ultrasound data, MRI data, or both, are displayed on a display monitor 128. Optionally, any of the power supplies are packaged together with each other, or with the controller, and may share some components. Alternatively, they are all housed separately. Optionally, the components of the controller which control the TMRI probe are housed separately from the components which control the TRUS probe. Alternatively, they are housed together. Optionally, some post-processing of MRI or ultrasound images is done in a physically separate location from the control and data receiving functions of controller 118.

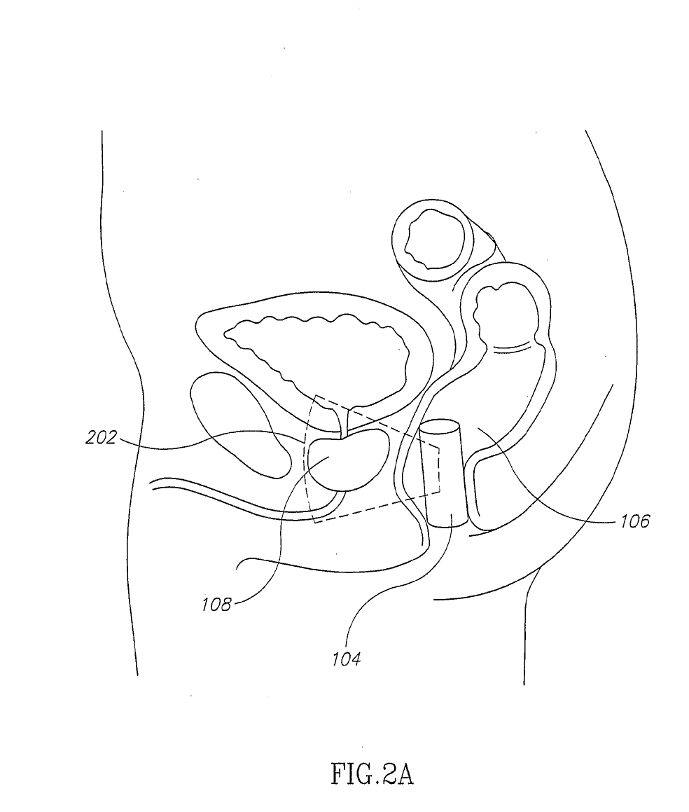

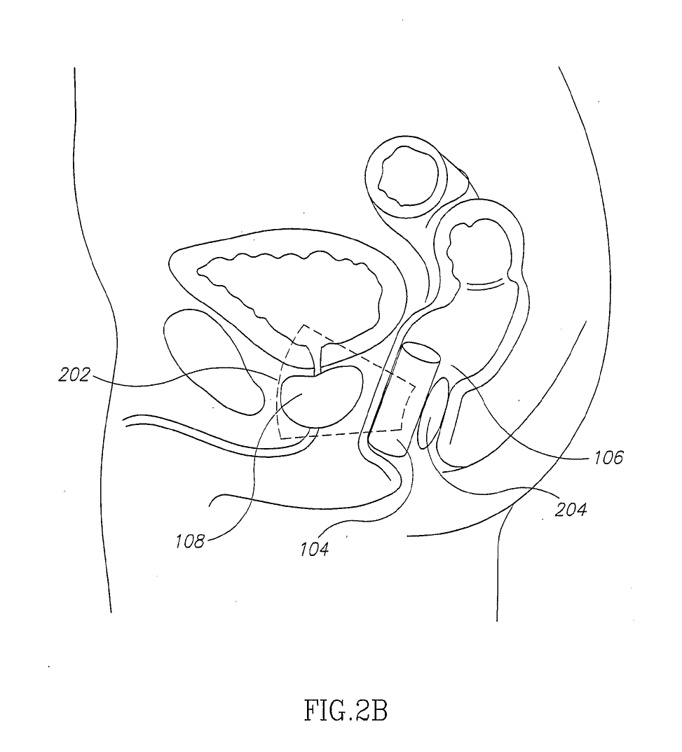

FIG. 2A shows a cylindrical TMRI probe 104, without a TRUS probe, positioned in the rectum 106. Although using a TRUS probe together with the TMRI probe has several potential advantages as described previously, use of the TMRI probe by itself, particularly in the positions shown in FIGS. 2A and 2B, may have the potential advantage that the center of the probe can be brought closer to the prostate, possibly improving the resolution or the SNR of the image, or decreasing the acquisition time, or having some combination of the these benefits. A field of view 202 (also referred to as the imaging region) of probe 104 optionally includes the entire prostate 108, as shown in FIG. 2A. For example, the field of view extends a distance 50 mm radially from the probe. Alternatively, the field of view may not include the entire prostate, and extends 30 mm from the probe, or 20 mm, or 10 mm. If the field of view extends radially out to a distance that is comparable to or greater than the radius of the probe, which is 15 mm for the probe shown in FIG. 2A, then at greater distances from the probe the resolution will be more coarse for a given SNR and acquisition time, or the SNR will be lower for a given pixel size and acquisition time. Other trade-offs are possible, for example using a longer acquisition time for regions that are further from the probe. Optionally, field of view 202 extends 45 degrees azimuthally, i.e. in the plane perpendicular to the longitudinal axis of probe 104. Alternatively, the field of view extends 90 degrees azimuthally, or 60 degrees, or 30 degrees, or 20 degrees. Optionally, field of view 202 extends 30 mm longitudinally. Alternatively, the field of view extends 50 mm longitudinally, or only 20 mm.

FIG. 2B shows another embodiment of the invention in which probe 104 is pushed closer to the prostate, using a balloon 204, located on the posterior side of probe 104. When probe 104 is inserted into rectum 106, balloon 204 expands, for example by filling it with air or with water through a tube (not shown). Expanded balloon 204 pushes against the posterior wall of the rectum, pushing probe 104 against the anterior wall of the rectum, where it is closer to prostate 108. As noted above, locating probe 104 closer to the prostate enables the MRI images to have one or more of higher resolution, higher SNR, and shorter acquisition time, and may also align the field of view so that it covers more of the prostate, if the field of view does not already cover the entire prostate. Balloon 204 is also optionally used with a combined TRUS and MRI probe, as in FIG. 1.

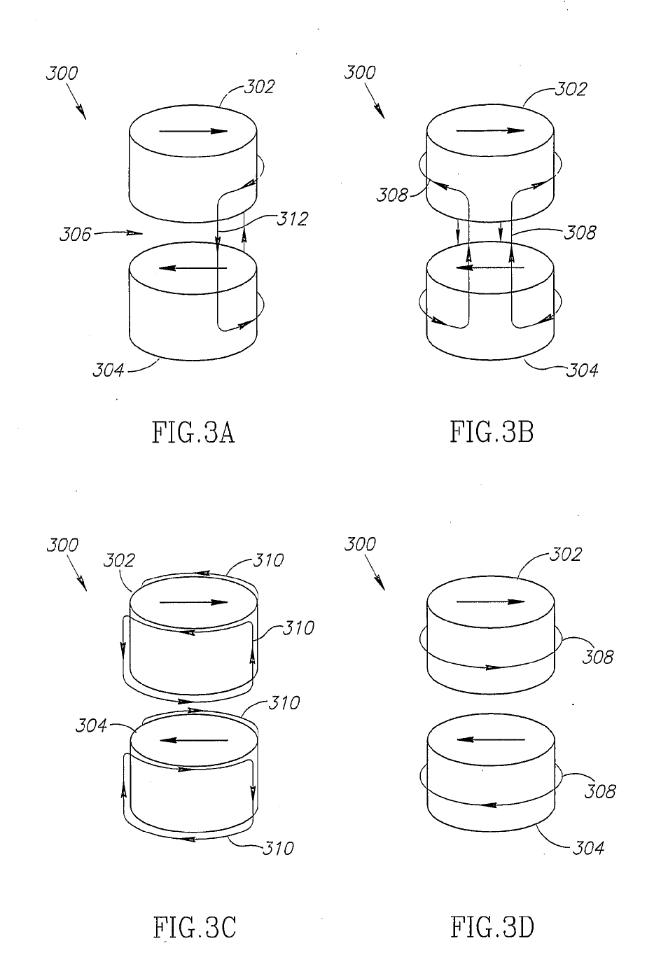

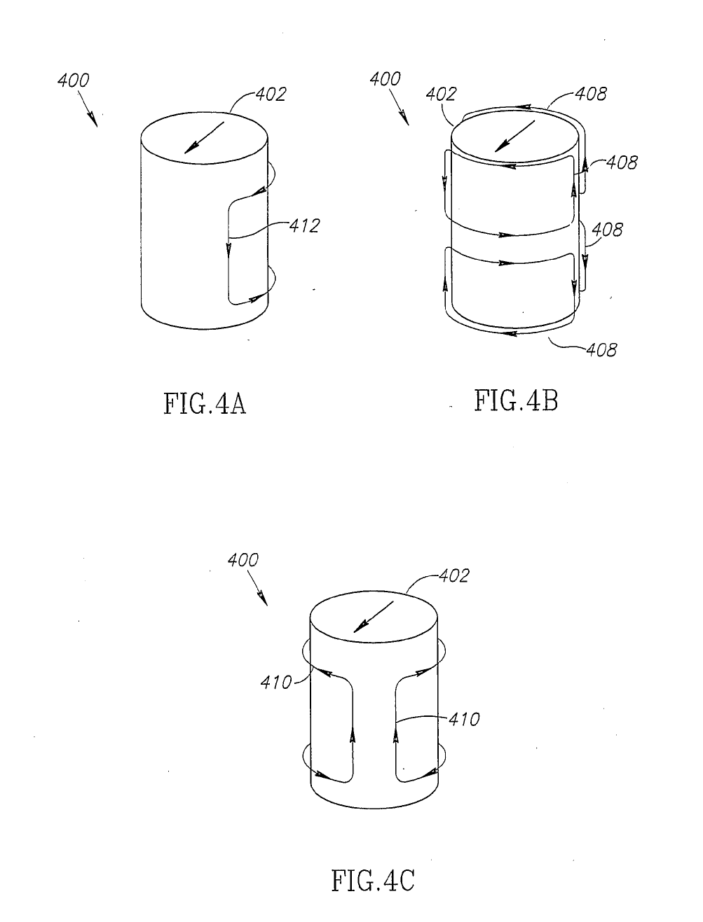

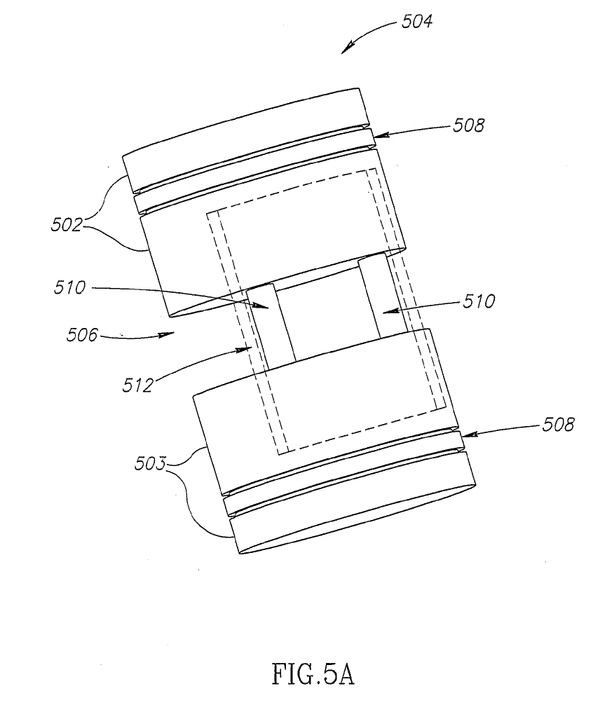

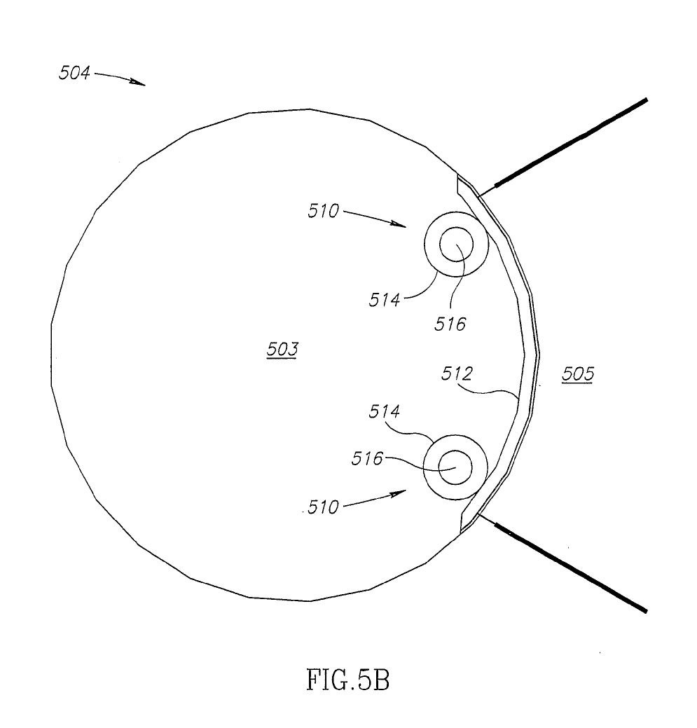

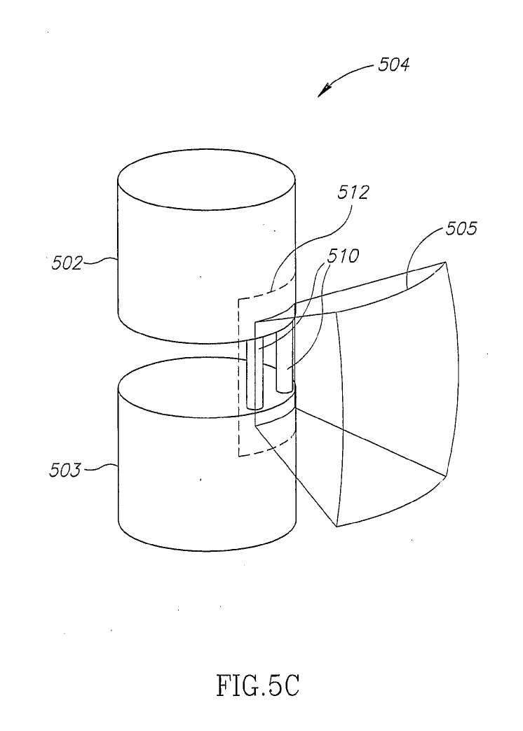

FIGS. 3A-3D are schematic views of a probe 300, which is optionally used, for example, for the TMRI probe shown in FIG. 1 or FIG. 2. Probe 300 comprises permanent magnets 302 and 304. Optionally, the magnets are rare earth magnets with a high energy product, for example neodymium-iron-boron magnets with an energy product of 50 MGOe. Using magnets of higher energy product has the potential advantage that, at a given distance from the probe relative to the probe diameter, the magnetic field will be higher, allowing higher SNR or higher resolution for the image. Each magnet is a cylinder, for example 50 mm long and 30 mm in diameter, arranged longitudinally, and there is optionally a gap 306 between the magnets, for example a gap of 10 mm. Alternatively each magnet is smaller, but at least 10 mm in diameter, or at least 20 mm in diameter. Magnets 302 and 304 are magnetized respectively in the +x and −x directions, defined by the arrows on the magnets in FIGS. 3A-3D, i.e. perpendicular to the longitudinal axis (the z-axis), and opposite to each other. The field of view of probe 104 is centered around the x-axis in one direction, for example the +x direction. FIG. 5C, below, shows a perspective view of a typical three-dimensional field of view 505 for a probe 504, which field of view is similar in shape, and similar in size relative to the probe, to the field of view of probe 104 in FIGS. 3A-3D and 4A-4C. Along the x-axis, the magnetic field produced by the magnets is oriented longitudinally, i.e. parallel to the z-axis, and this is approximately true throughout the field of view. On the x-axis, for the example given, the field is about 0.35 tesla at the probe surface, and falls to about 0.01 tesla at a distance of 50 mm from the probe surface. The field gradient is about 1 tesla/meter or more, in the field of view. Optionally, the field is at least 0.35 tesla at at least one point on the surface of the probe, at least 0.01 tesla at at least one point at a distance of 50 mm from the probe surface, and the gradient is at least about 1 tesla/meter, at at least one point 50 mm from the probe surface.

Optionally, probe 300 also includes a set 308 of z-gradient coils and/or a set 310 of φ-gradient coils, where φ is the azimuthal angle. For clarity, the z-gradient coils are shown in FIG. 3B, and the φ gradient coils are shown in FIG. 3C, but optionally both sets of gradient coils (together with the RF coil described below) are present together. An alternative configuration for the z gradient coils is shown in FIG. 3D. In FIGS. 3A-3D, the x-axis, which is the axis that the field of view is centered around, is horizontal, the z-axis (the longitudinal direction) is vertical, and the y-direction (the azimuthal direction in the field of view) is normal to the plane of the drawing.

There is also an optional RF coil 312 shown in FIG. 3A, or another type of RF antenna, optionally disposable. Since it is potentially advantageous to have the RF coil directly in contact with the rectal wall, in order to make it as close as possible to the prostate, making the RF coil disposable can ensure that is sterile and/or in good condition when it is used. The RF field is predominantly in the x-direction, and therefore substantially perpendicular to the static magnetic field, over most of the field of view, making efficient use of the static and RF magnetic fields.

FIG. 4A shows-an-MRI probe 400 with an alternative magnet configuration instead of two magnets magnetized in opposite directions perpendicular to the longitudinal axis, as in FIGS. 3A-3D, there is a single magnet 402, magnetized perpendicular to the longitudinal axis, for example in the y-direction, as shown in FIG. 4A. The field of view is centered around the x-axis. The static magnetic field is largely in the y-direction (the azimuthal direction) over most of the field of view. An RF magnetic field, produced by an RF coil 412, is predominantly in the x-direction, as in FIGS. 3A-3D, so again the RF magnetic field and static magnetic field are substantially perpendicular to each other, and efficient use is made of the fields. Like probe 300, the probe 400 has z-gradient coils 408 (shown only in FIG. 4B for clarity) and φ-gradient coils 410 (shown only in FIG. 4C, for clarity).

Although the gradient coils and RF coils in FIGS. 3A-3D and 4A-4C are shown placed outside the magnets, they are optionally at least partly located in grooves carved into the outside surface of the magnets, or in slots that are largely surrounded by the magnets. The RF coils, however, are optionally not largely surrounded by any of the magnets, unless the skin depth of the magnet at the RF frequency is greater than the magnet dimensions, or unless the magnet is laminated. The same is true of the gradient coils, if the skin depth at the gradient pulse frequency is not greater than the magnet dimensions.

Although the gradient coils are drawn as thin lines in FIGS. 3B-3D and 4B-4C, the conductors optionally have cross-sections that have dimensions comparable to the coil and magnet dimensions, in order to minimize the ohmic power needed to produce a given field gradient. Where any of FIGS. 3B-3D and 4B-4C show two coils with current flowing in a same direction, the two coils are optionally replaced by a single coil, located for example between the two coils, with current flowing in the same direction as in the two coils.

FIG. 5A is a different schematic view of an exemplary probe 504, for example the TMRI probe shown in FIG. 1 or FIG. 2. FIG. 5B is a cross-sectional view through a gap defined therein. This probe is a scaled-up version of the miniature 2D imaging probe described in U.S. Pat. No. 6,704,594, approximately 15 times larger in linear dimensions, with an additional z-slice separation capability, using z gradient coils, to facilitate 3D imaging. Probe 504 comprises permanent magnets 502 and 503. Optionally, the magnets are rare earth magnets with a high energy product, for example neodymium iron magnets with an energy product of 50 MGOe. Each magnet is a cylinder 50 mm long and 30 mm in diameter, arranged longitudinally, and there is optionally a gap 506 between the magnets, for example a gap of 10 mm. The magnets are magnetized respectively in the +x and −x directions, i.e. perpendicular to the longitudinal axis (the z-axis), and opposite to each other. A field of view 505 of probe 504 is centered around the x-axis in one direction, for example the +x direction, and shown in FIGS. 5B and 5C.

Probe 504 also includes one or more than one RF coil 512, or another type of RF antenna, optionally disposable. In FIG. 5A, RF coil 512 is shown as a dotted rectangle, so that an underlying set 510 of φ-gradient coils can be seen. However, coil 512 will typically be solid and opaque. Optionally, coil 512 is provided in a recess of probe 504, as shown in FIGS. 5B. Alternatively, coil 512 may be located outside of the probe, for example as an extra layer, facilitating its removal and replacement. The probe may be covered with a (optionally disposable) layer, such as a condom, and RF coil 512 is outside this layer. In an exemplary embodiment of the invention, the RF coil is a flat structure of about 30 mm in height, about 1 mm thick and spanned across an angle of about 90 degrees around the probe structure. Smaller or larger angles are possible, for example, between 30 or 45 degrees and 120 degrees, depending on the desired field of view. The coil is located as close as possible to the field of view to increase SNR.

The magnetic field outside the probe, which is highly non-homogenous, enables 1D spatial encoding of the examined sample in the radial direction. To facilitate 3D spatial encoding of the sample voxels, the probe contains an additional two sets of gradient coils, a set 508 of z-gradient coils, and set 510 of φ-gradient coils. Set 508 of z-gradient coils is optionally based on a standard Maxwell pair. Set 510 of φ-gradient coils is optionally not a conventional one. Its construction, as shown in the top view of FIG. 5B, is based on two solenoids 514 each wound around a high saturation iron powder core 516, for example as described in U.S. Pat. No. 6,704,594. These solenoids optionally generate opposing fields in the z direction that cancel each other at φ=0, and the field in the z-direction is a linear function of φ near φ=0. The two sets of gradient coils, together with the radial gradient in the field produced by the permanent magnets, optionally allow the probe to produce a three-dimensional image, or a series of two dimensional images which can be combined to form a three-dimensional image, using standard techniques of MRI.

Specific imaging sequences that may be employed to obtain the 3D image are Ultra-fast RARE or similar methods (Mag. Res. Med. 27, p. 142-164 1992, the disclosure of which is incorporated herein by reference), with constant gradient applied to one axis (by the permanent magnet), or the imaging sequences described in U.S. Pat. No. 6,600,319, the disclosure of which is incorporated herein by reference, and in U.S. Pat. No. 6,704,594, with an additional z-slice selection pulse. Optionally, a modified form of a CPMG pulse sequence, with short echo time and a large number of echoes, is used to obtain a reasonable SNR in the highly inhomogeneous magnetic field, as described, for example, in various patents, patents applications and/or publications mentioned herein. The CPMG sequence is a spin-echo sequence, in which an initial 90-degree RF pulse is followed by a train with a plurality (sometimes a large number) of 180-degree RF pulses (each one 90 degrees out of phase with the 90-degree RF pulse), and echoes are detected between the 180-degree RF pulses. In the imaging CPMG pulse sequence used in conventional MRI, gradient pulses are typically applied between each of the 180-degree RF pulses in the pulse train. This has the potential advantage that the entire k-space may be covered much faster, but the high repetition rate of the gradient pulses may produce excessive heating due to ohmic loses in the gradient coils. In the above mentioned patents, which describe self-contained intravascular MRI probes with rather large pixel size relative to the probe size, this problem is optionally avoided by not using gradient coils at all, but by rotating and translating the probe in order to obtain azimuthal and longitudinal resolution. For a prostate probe, which optionally is designed to have a pixel size much smaller than the probe size, however, gradient coils are useful.

Other families of imaging sequences avoid this heating problem by using the technique described in U.S. Pat. No. 5,493,225 to Crowley, or a similar technique such as described in Journal of Magnetic Resonance 166 (2004), 228-235, the disclosures of which are incorporated herein by reference. In such techniques, the gradient pulses are applied only once, for each pulse train, at the beginning, and the pulse train is then repeated using a different relative phase (for example, zero degrees) between the 90-degree RF pulse and the 180-degree RF pulses. In choosing an imaging sequence to use, the advantage of these methods in avoiding gradient coil heating should be balanced in turn by the much lower k-space coverage rate and the requirements for image pixel size and imaging acquisition time.

The z gradient coils, a Maxwell pair, optionally provide a gradient of about 50 milli-tesla per ampere-meter, and have a resistance of about 20 ohms, and similar numbers may apply to the φ gradient coils. To keep the ohmic power of the gradient coils reasonably low (when considering imaging sequences with a gradient pulse applied between each of the 180 degree RF pulses), for example under 250 watts average power (optionally with active cooling), the gradient pulses are optionally sufficiently long, for example about 0.2 milliseconds, and the time between 180-degree RF pulses (and between successive echoes) is, for example, about 0.5 milliseconds. However, to avoid having the excited nuclei lose too much coherence due to diffusion, it may be desirable to make the time between 180-degree RF pulses somewhat shorter, for example about 0.25 milliseconds. The other imaging sequences, in which a gradient pulse is applied only at the beginning of each 180-degree RF pulse train, result in much lower heating and may apply much shorter gradient pulses, on the order of 50 microseconds or less. All of these numbers are merely representative, and may vary considerably depending on the design of the magnets, gradient coils, RF coil, and pulse sequence.

With the above imaging sequences one can achieve, for example, voxels which are between 1.5 mm and 4 mm wide in the radial and azimuthal directions, depending on how far they are from the probe, and between 3 mm and 4 mm wide in the longitudinal direction. Alternatively, the voxels are larger than this, or smaller than this. Optionally, the voxels are larger at a greater distance from the probe, in order to avoid having lower SNR at a greater distance from the probe.

The time requirement to acquire the image depends not only on the k-space coverage rate but also upon the SNR of the system. The RF magnetic field is optionally at least 0.0025 tesla everywhere in the field of view. The sensitivity of the RF coil may be as high as 4 microtesla per ampere, for example, if the RF coil has an optimized shape consisting of about 20 windings of 0.2 mm diameter copper wire, with a resistance of 0.01 ohms, and there is an optimized tuning capacitor. Optionally, the RF coil produces a field of at least 4 microtesla per ampere of current, at at least one point 50 mm from the surface of the probe. With these numbers, and 1.5 mm×1.5 mm×4 mm voxels, the SNR for a single pulse train lasting 2 milliseconds may be about 0.004. Averaging over 375,000 pulse trains during a 15 minute acquisition time, the SNR would be about 2.5. These numbers are greatly improved when using increased voxel size and/or acquiring signal at locations that are closer than 50 mm from the probe surface.

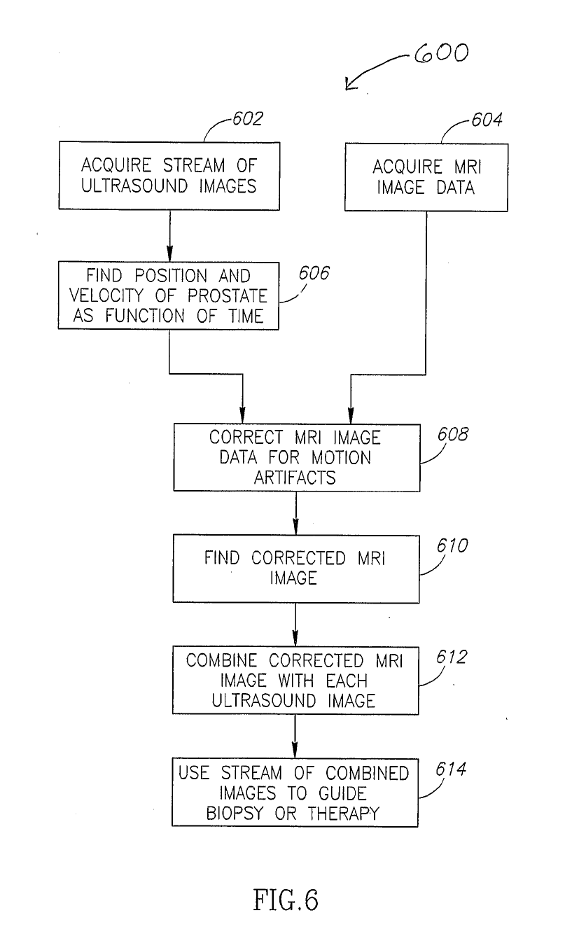

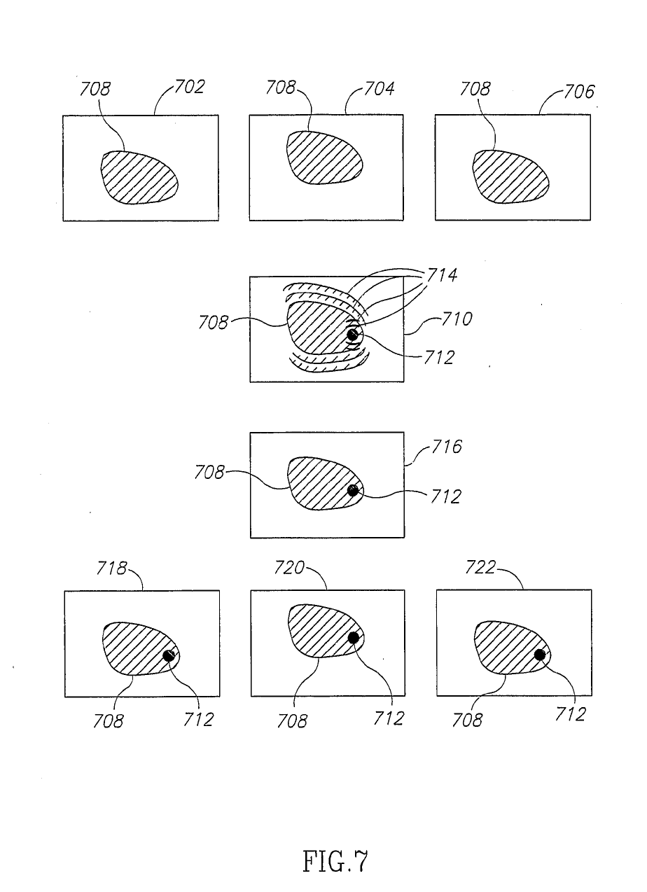

FIG. 6 shows a flowchart 600, describing a method of using combined ultrasound and MRI imaging data to reconstruct images of the prostate that have more information than would be obtained with either the ultrasound or the MRI data alone. The reconstruction is done, for example, by controller 118 in FIG. 1, or by another computer, and the reconstructed images are optionally displayed on display monitor 128, in real time or after post-processing. FIG. 6 will be described together with FIG. 7, which shows images reconstructed at different stages in the method shown in FIG. 6. The ultrasound data is acquired, for example, with TRUS probe 102 in FIG. 1, the MRI data is acquired with TMRI probe 104, and the image reconstruction is done, for example, by controller 118, or by another computer.

At 602, a stream of ultrasound images is obtained using data from the ultrasound probe, in real time. Images 702, 704, and 706 in FIG. 7 schematically represent such a time stream of ultrasound images. In fact, there may be far more than three images in the stream. Prostate 708 is visible in each of the images, but typically moves around from one image to the next, due to the normal slight motion of the prostate in the body. The distance which the prostate moves is exaggerated in FIG. 7, for clarity.

At the same time, at 604, MRI data is obtained from the MRI probe. Because the acquisition time for the ultrasound images is much shorter than the acquisition time for an MRI image, several ultrasound images are obtained during the time data is acquired for one MRI image. A clock in controller 118, for example, synchronizes the acquisition of the MRI data with the acquisition of the ultrasound data, so that it is possible to find out which ultrasound image was being acquired at the time of any received echo in the MRI data.

Due to the motion of the prostate, an MRI image 710 reconstructed from the raw MRI data, shows motion artifacts 714. As is well known, due to the nature of MRI image reconstruction, MRI motion artifacts may be more severe than just a blurring of the image corresponding to the change in position of the prostate during the acquisition time. MRI image 710, unlike ultrasound images 702, 704 and 706, shows a tumor 712. This is done, for example, by using diffusion weighting in acquiring the MRI data, since prostate tumors generally have a different diffusion coefficient than healthy prostate tissue. Alternatively, one or more other kinds of MRI weighting, such as T₁ or T₂ weighting, are used, in addition to or instead of diffusion weighting, to distinguish tumor 712 from the rest of prostate 708. But because of the motion artifacts, the image quality of image 710 is poor, and it may not even be possible to see tumor 712 in image 710. But it may also be difficult or impossible to see tumor 712 on the ultrasound images, because the sound speed and acoustic impedance of ultrasound may not differ appreciably in a tumor and in healthy prostate tissue.

At 606, the position and velocity of the prostate as a function of time is calculated, using the ultrasound image stream represented by images 702, 704 and 706. At 608, this information on the motion of the prostate is used to correct for motion artifacts in the MRI data, and at 610, an MRI image 716 is reconstructed, relatively free, at least, of motion artifacts. In image 716, tumor 712 is more clearly visible than in image 710.

Alternatively, instead of using the ultrasound image stream, a sequence of MRI images is acquired using very short acquisition times. Although these images will have low SNR, and are not corrected for motion artifacts, they may still be clear enough to be used for calculating the position and velocity of the prostate at each time. These low SNR images are then combined to produce an MRI image with high SNR, which is corrected for motion artifacts using the information on position and velocity of the prostate. This alternative can be accomplished using an MRI rectal probe, even without an ultrasound probe.

If the images are being acquired during the performance of a biopsy, or during invasive therapeutic treatment such as implantation of radiation sources, or ablation, then the image may include an instrument such as a biopsy needle (not shown), which is also moving, and not generally at the same velocity as the prostate is moving. In this case, optionally, at 606, the position and velocity of the instrument are found, in addition to the position and velocity of the prostate, using the stream of ultrasound images. Also, at 608, the information is used to correct for both the motion of the prostate and the (generally different) motion of the instrument, in the MRI data.

At 612, corrected MRI image 710 is combined with each of the images in the time stream of ultrasound images, in each case correcting for the changing position of the prostate in the ultrasound images. If there is an instrument such as a biopsy needle in the images, then the changing position of the instrument is also corrected for, when combining MRI image 710 with the ultrasound images. The result is shown schematically in images 718, 720, and 722. The motion of the prostate may be followed in real time, as well as the motion of any instrument that is present. But, in contrast to ultrasound images 702, 704 and 706, tumor 712 is visible in combined images 718, 720, and 722.

Optionally, at 614, the combined image stream is viewed in real time, for example on display monitor 128 in FIG. 1, by a physician performing a biopsy or invasive therapy. Because the continuously changing position of the tumor and the instrument are visible in the combined image stream in real time, the combined image stream is more useful for guiding the biopsy or therapy than either ultrasound images or MRI images alone.

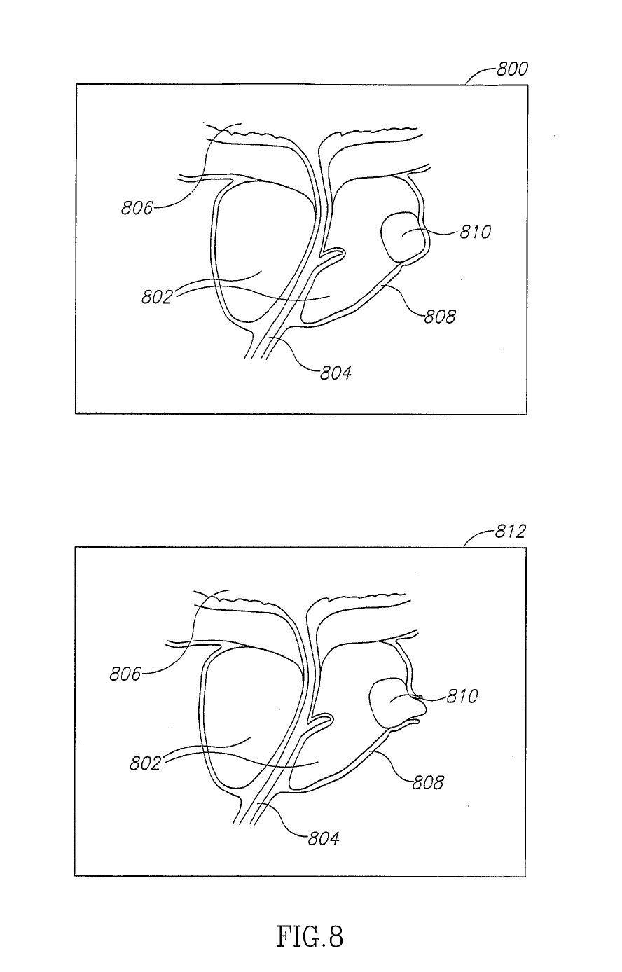

In addition to the synergistic effect of combining MRI and ultrasound imaging data in guiding a biopsy or therapy, MRI and ultrasound also complement each other in staging prostate cancer. FIG. 8 shows two images, an image 800 and an image 812, reconstructed using both MRI and ultrasound data, for example using the method shown in flowchart 600, or even simply by overlaying an MRI image and an ultrasound image. Image 800 shows a side cross-sectional view of a prostate 802. A urethra 804 and bladder 806 are also visible in the image. Prostate 802 is surrounded by a prostatic capsule 808, and there is a cancerous tumor 810 adjacent to membrane 808 in a posterior part of the prostate. Tumor 810 is still in stage T2, because it has not yet broken through capsule 808. This can be seen in the combined MRI and ultrasound image, but it would be difficult to tell this from either an ultrasound image or an MRI image alone. Tumor 810 can be distinguished from healthy prostate tissue in an MRI image, by, for example, the difference in the diffusion coefficients that characterize the two tissues. But tumor 810 would be difficult to see in an ultrasound image, since the tumor and the healthy prostate tissue do not have very different responses to sound waves. Prostatic capsule 808, on the other hand, is relatively easy to see in an ultrasound image, but is not easy to detect in an MRI image, since it is very thin. Image 812 shows tumor 810 in stage T3, after it has broken through capsule 808. Knowing whether prostate cancer is in stage T2 or T3 is important in deciding how to treat it.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. A rectal probe adapted for ultrasound and magnetic resonance imaging of the prostate, comprising:
a) an ultrasound imaging probe;
b) an MRI probe comprising a first magnetic field source for creating a static magnetic field in an MRI imaging region outside the rectal probe, a second magnetic field source for creating a time-varying magnetic field which excites nuclei in the MRI imaging region, and a receiver for receiving NMR signals from the excited nuclei and generating MRI imaging data indicative thereof; and
c) a link joining the ultrasound probe and the MRI probe.

2. A rectal probe according to claim 1, wherein the link is flexible, thereby allowing a direction of orientation of the MRI probe to vary relative to a direction of orientation of the ultrasound probe.

3. A rectal probe according to claim 1, wherein the MRI probe has a longitudinal axis, and the static magnetic field is substantially inhomogeneous, defined as monotically decreasing with increasing distance from the longitudinal axis, outside the MRI probe, in all directions.

4. A rectal probe according to claim 1, wherein the first magnetic field source is a permanent magnet, and the magnetic field is at least 0.35 tesla at at least one point on the surface of the MRI probe.

5. A rectal probe according to claim 1, wherein the first magnetic field source is a permanent magnet, and the magnetic field is at least 0.01 tesla at at least one point at a distance 50 mm from the surface of the MRI probe.

6. A rectal probe according to claim 1, wherein the first magnetic field source is a permanent magnet, and the magnetic field has a gradient of at least about one tesla per meter, at at least one point 50 mm from the surface of the MRI probe.

7. A rectal probe according to claim 1, wherein the second magnetic field source is a coil which produces a magnetic field of at least 4 micro-tesla per ampere of current, at at least one point at a distance 50 mm from the surface of the probe.

8. An imaging system comprising:
a) a rectal probe according to claim 1; and
b) an RF power supply which supplies power to the second magnetic field source;
wherein the RF power supply is capable of supplying the second magnetic field source with sufficient power at a great enough frequency range to simultaneously excite nuclei in the imaging region to generate NMR signals having a frequency bandwidth of more than 5% of their mean frequency, and wherein the static magnetic field monotonically decreases with increasing radial distance from the outer surface of the MRI probe, everywhere outside the outer surface of the MRI probe.

9. An imaging system according to claim 8, wherein the NMR signals have a frequency bandwidth of more than 10% of their mean frequency.

10. An imaging system according to claim 9, wherein the NMR signals have a frequency bandwidth of more than 20% of their mean frequency.

11. An imaging system comprising:
a) a rectal probe according to claim 1; and
b) an RF power supply which supplies a given power to the second magnetic field source;
wherein the MRI probe, when supplied with said given power by the RF power supply, is capable of producing MRI imaging data with sufficient spatial resolution and signal to noise ratio to be capable of revealing a tumor 5 mm in diameter located anywhere in the prostate.

12. An imaging system comprising:
a) a rectal probe according to claim 1; and
b) an RF power supply which supplies a given power to the second magnetic field source;
wherein the second magnetic field source, when supplied with said given power by the RF power supply, is capable of creating a time varying magnetic field of at least 0.0025 tesla at at least one point at a distance of 50 mm from the surface of the MRI probe.

13. An imaging system comprising:
a) a rectal probe according to claim 1;
b) an RF power supply which supplies power to the second magnetic field source; and
c) a controller which controls the RF power supply to produce a timed sequence of RF pulses;
wherein the controller is adapted to control the RF power supply to produce the sequence of RF pulses with a repetition rate greater than one pulse every 0.5 milliseconds and less than one pulse every 0.25 milliseconds, and the RF power supply is capable of producing the sequence of RF pulses at said repetition rate.

14. An imaging system comprising:
a) a rectal probe according to claim 1, with a longitudinal axis, also including at least one gradient coil which produces a magnetic field gradient in the direction of the longitudinal axis, or in an azimuthal direction around the longitudinal axis;
b) a gradient coil power supply which supplies current to the gradient coil; and
c) a controller which controls the gradient coil power supply to produce a timed sequence of gradient pulses;
wherein the controller is adapted to make the gradient pulses at least about 0.2 milliseconds long.

15. A method of imaging the prostate, the method comprising:
a) inserting a rectal probe according to claim 1 into a rectum;
b) acquiring at least one ultrasound image of the prostate, using the ultrasound probe;
c) acquiring MRI imaging data of the prostate using the MRI probe; and
d) reconstructing an image of the prostate from the MRI imaging data, using an image reconstruction procedure;
wherein an adjustment is made to one or both of the MRI probe and the image reconstruction procedure using information from the ultrasound image.

16. A method according to claim 15, wherein the adjustment causes the MRI imaging region to correspond more closely to the prostate or to a desired portion of the prostate.

17. A method according to claim 15, wherein the at least one ultrasound image comprises a plurality of ultrasound images acquired at different times between the beginning and end of acquisition of the MRI image, and the adjustment corrects for motion of the prostate occurring between the beginning and end of acquisition of the MRI image.

18. A method according to claim 15, wherein the information from the ultrasound image comprises a Doppler shift, and the adjustment corrects for motion of the prostate occurring during acquisition of the MRI image.

19. A method of diagnosing the stage of prostate cancer, comprising:
a) inserting a rectal probe according to claim 1 into a rectum;
b) acquiring an ultrasound image of the prostate using the ultrasound probe;
c) acquiring an MRI image of the prostate using the MRI probe;
d) finding prostate cancer on the MRI image; and
e) examining the ultrasound image to determine whether or not the prostatic capsule is intact in the vicinity of the prostate cancer found on the MRI image.

20. A method according to claim 19, wherein acquiring an MRI image comprises acquiring a diffusion-weighted MRI image.

21. A method of performing one or both of a biopsy and therapy on the prostate, comprising:
a) inserting a rectal probe according to claim 1 into a rectum;
b) acquiring at least one ultrasound image of the prostate, using the ultrasound probe;
c) acquiring MRI imaging data of the prostate using the MRI probe;
d) reconstructing an MRI image of the prostate from the MRI imaging data, using an image reconstruction procedure; and
e) performing one or both of a biopsy and therapy on the prostate, guided in real time by the ultrasound image and the MRI image.

22. A method of diagnosing prostate cancer, comprising:
a) inserting into the rectum an MRI probe with a longitudinal axis, the MRI probe comprising a first magnetic field source for creating a substantially inhomogeneous static magnetic field in an MRI imaging region outside the probe, a second magnetic field source for creating a time-varying magnetic field which excites nuclei in an extended sub-region of the MRI imaging region, and a receiver for receiving the NMR signals from the excited nuclei and generating MRI imaging data indicative thereof;
b) acquiring a diffusion-weighted MRI image of the prostate using the MRI probe; and
c) finding prostate cancer on the MRI image by using the difference in diffusion rate between cancerous and healthy tissue.

23. An imaging system comprising:
a) an MRI rectal probe adapted for imaging of the prostate, the probe having a longitudinal axis and comprising a first magnetic field source for creating a static magnetic field in an MRI imaging region outside the probe, a second magnetic field source for creating a time-varying magnetic field which excites nuclei in the MRI imaging region, and a receiver for receiving NMR signals from the excited nuclei and generating MRI imaging data indicative thereof; and
b) an RF power supply which supplies power to the second magnetic field source;
wherein the RF power supply is capable of supplying the second magnetic field source with sufficient power at a great enough frequency range to simultaneously excite nuclei in the imaging region to generate NMR signals having a frequency bandwidth of more than 5% of their mean frequency, and wherein the static magnetic field monotonically decreases with increasing radial distance from the longitudinal axis, everywhere outside the probe.

24. An MRI probe having a longitudinal axis, the probe comprising:
a) a first magnetic field source for creating a static magnetic field in an MRI imaging region outside the probe, which static magnetic field monotonically decreases with increasing distance from the longitudinal axis to the probe, everywhere outside the probe;
b) a second magnetic field source for creating a time-varying magnetic field which excites nuclei in the MRI imaging region;
c) a receiver for receiving the NMR signals from the excited nuclei and generating MRI imaging data thereof; and
d) at least one z gradient coil which produces a magnetic field gradient in the direction of the longitudinal axis.

25. A probe according to claim 24, of a size suitable for use as a rectal probe for imaging the prostate.
